(19) United States
(12) Patent Application Publication
(10) Pub. No.: US 2022/0018837 A1
(43) Pub. Date: Jan. 20, 2022

(54) Method for the Detection of Surface-Mounted Biological Materials and Pathogens

(71) Applicant: Nanoco Technologies Ltd., Manchester (GB)

(72) Inventor: Imad Naasani, Manchester (GB)

(21) Appl. No.: 17/369,691

(22) Filed: Jul. 7, 2021

Related U.S. Application Data
(60) Provisional application No. 63/053,367, filed on Jul. 17, 2020.
(60) Provisional application No. 63/057,647, filed on Jul. 28, 2020.

(51) Int. Cl. G01N 33/569; G01N 33/543; G01N 21/64

(52) U.S. Cl. CPC G01N 33/56983; G01N 33/54346; G01N 2021/6441; G01N 2333/165; G01N 21/6428

(57) ABSTRACT

Methods and compositions for the detection of surface-mounted pathogens are described herein. Compositions include preparations comprising quantum dot-ligand conjugates, wherein the ligands target a specific pathogen to form a quantum dot-pathogen complex. Methods include the use of the preparations comprising the quantum dot-ligand conjugates. The preparations may be applied to a surface for the detection of a surface-mounted pathogen thereon via fluorescence, which may be detected by the naked eye or a simple fluorescence camera.

### CROSS-REFERENCE TO RELATED APPLICATIONS

This application claims the benefit of U.S. Provisional Application No. 63/053,367 filed Jul. 17, 2020 and U.S. Provisional Application No. 63/057,647 filed Jul. 28, 2020, the entire contents of which are incorporated by reference herein.

### BACKGROUND OF THE INVENTION

### 1. Field of the Invention

The invention relates to the detection of surface-mounted biological materials and pathogens, including cells from tissue specimens and fluids of humans, animals, plants, and pathogens such as prions, viruses, microorganisms, fungi and algae. More particularly, the invention relates to the detection of surface-mounted biological samples and pathogens using fluorescent semiconductor quantum dots.

### 2. Description of the Related Art including Information Disclosed under 37 CFR 1.97 and 1.98

Conventional methods for the detection of surface-mounted biological systems and microorganisms, such as isolated cellular tissues and pathogens such as prions, viruses, bacteria and fungi are complicated, time-consuming and expensive. For example, methods based on the polymerase chain reaction (PCR) are expensive, prone to false positive results, and require extensive training. Other methods may employ microscopy and/or the detection of markers using immunoassays that involve expensive multi-step processes. Thus, there is a need for a simple, robust method for the detection of pathogens on surfaces, for use at point-of-care facilities, fields of exposure, and in households, not only for use in medical applications, but also for use in veterinary settings and agricultural practices (e.g. farming).

### BRIEF SUMMARY OF THE INVENTION

Herein, methods for the detection of surface-mounted biological systems in tissue samples or bodily fluids using quantum dots (QDs) are described. The QDs are conjugated with ligands having an affinity for a target pathogen. Examples of target pathogens may include, but are not restricted to: prions, viruses, and microorganisms such as, but not limited to, bacteria, fungi, algae, and parasites. Examples of tissue samples may include, but are not restricted to cells such as subtypes of white blood cells and isolated cells from bodily fluids, and cellular tissues. In some embodiments of the disclosure, the target pathogen to be detected is a strain or variant of coronavirus, such as SARS-CoV-2.

Once the QDs are functionalised with targeting ligands, they may be formulated into a preparation that can specifically bind to a corresponding target to form a QD-target (or QD-pathogen) complex that, when irradiated with a light source, may be visible to the naked eye or with a simple fluorescence camera. The specificity of this approach may be enhanced by using QDs emitting at two or more different wavelengths (colours) that can bind to the same target pathogen or biological structure. When using two or more colours of QDs, the emitted light from each type (colour) of QDs will overlap to produce a unique combination of emitted light that is specific and only attainable in the presence of a specific target pathogen or biological systems.

Alternatively, a single type (colour) of QDs may be conjugated with a first type of targeting ligand bound covalently to the QD surface and a second type of targeting ligand physically attached to (adsorbed on) the QD surface, and concurrently, the second type of targeting ligand may be covalently linked with a QD quencher. In the presence of a corresponding target, the first type of targeting ligand enables the QDs to latch on to the target, while the second type of targeting ligand becomes detached from the QD and binds to a different target, enabling the QDs of the resulting QD-target (or QD-pathogen) complex to fluoresce upon excitation by a light source.

A preparation comprising QD-ligand conjugates may be in any suitable form including, but not limited to, a spray, a gel, or a lateral flow assay.

In accordance with various aspects of the disclosure, methods for detecting a pathogen in a tissue sample or bodily fluid are provided. The methods comprise providing a composition comprising a population of quantum dot (QD)-ligand conjugates, the population of QD-ligand conjugates comprising a first population of QD-ligand conjugates configured to absorb light from a UV- or blue-emitting light source and emit light at a first wavelength, and a second population of QD-ligand conjugates configured to absorb light from a UV- or blue-emitting light source and emit light at a second wavelength; contacting the composition comprising the population of quantum dot (QD)-ligand conjugates with a tissue sample or a bodily fluid containing a pathogen to bond one or more of the QD-ligand conjugates to the pathogen and form QD-pathogen complexes; and subjecting the QD-pathogen complexes to irradiation with a light source. In some instances, the first wavelength of light is in the red portion of the electromagnetic spectrum and the second wavelength of light is in the green portion of the electromagnetic spectrum. In some instances, the first wavelength of light is in the green portion of the electromagnetic spectrum or the second wavelength of light in in the red portion of the electromagnetic spectrum. In some instances, the tissue sample is a population of white blood cells. In some instances, the tissue sample is a population of cells isolated from a bodily fluid. In some instances, the pathogen is selected from the group consisting of prions, viruses and microorganisms. In some instances, the pathogen is a coronavirus. In some instances, the pathogen is a coronavirus, wherein the coronavirus is SARS-CoV-2 or a strain or variant thereof. In some instances, the pathogen is a microorganism selected from the group consisting of bacteria, fungi, algae and parasites. In some instances, a QD-ligand conjugate of the first population comprises a QD conjugated to anti-SARS-CoV-2 spike S1 protein. In some instances, a QD-ligand conjugate of the second population comprises a QD conjugated to anti-SARS-CoV-2 nucleocapsid protein. In some instances, a QD-ligand conjugate of the first population comprises a QD conjugated to anti-SARS-CoV-2 spike S1 protein and a QD-ligand conjugate of the second population comprises a QD conjugated to anti-SARS-CoV-2 nucleocapsid protein. In some instances, the QD-ligand conjugates of the first population are made of red-emitting conjugates conjugated to anti-SARS-CoV-2 spike S1 protein and the QD-ligand conjugates of the second population are made of green-emitting conjugates conjugated to anti-SARS-CoV-2 spike S2 protein. In some instances, the QD-ligand conjugates of the first population are made of green-emitting conjugates conjugated to anti-SARS-CoV-2 spike S1 protein and the QD-ligand conjugates of the second population are made of red-emitting conjugates conjugated to anti-SARS-CoV-2 spike S2 protein. In some instances, yellow light is produced when QD-ligand conjugates of the first population and QD-ligand conjugates of the second population are bound to a pathogen such as a coronavirus, in some instances, SARS-CoV-2 virus, and subjected to irradiation with a UV-of blue-emitting light source. In some instances, the composition is a gel formulation comprising the population of QD-ligand conjugates and a thickening agent. In some instances, the gel formulation further comprises at least one of a buffer and a gelling agent. In some instances, the population of QD-ligand conjugates is lyophilised prior to mixing with the tissue sample or bodily fluid. In some instances, the composition is a solution comprising the population of quantum dot (QD)-ligand conjugates and a solvent. In some instances, the solution further comprises at least one of a surfactant, a buffer and a stabilizing agent. In some instances, contacting the composition comprising the population of quantum dot (QD)-ligand conjugates with the tissue sample or the bodily fluid containing the pathogen comprises applying the composition on a surface where the tissue sample or bodily fluid is located. In some instances, methods according to the above can be performed using a lateral flow device. In some instances, the QD-ligand-conjugates can have QDs that emit light in the yellow portion of the electromagnetic spectrum. In some instances, the QD-ligand-conjugates can have QDs that emit light in the orange portion of the electromagnetic spectrum. In some instances, the QD-ligand-conjugates can have QDs that emit light in the violet portion of the electromagnetic spectrum. In some instances, the QD-ligand-conjugates can have QDs that emit light in the infra-red portion of the electromagnetic spectrum.

In accordance with various aspects of the disclosure, additional methods for detecting a pathogen in a tissue sample or bodily fluid are provided. Such additional methods comprise providing a composition comprising a quantum dot (QD)-ligand conjugate, the QD-ligand conjugate comprising a first type of ligand covalently bound to the quantum dot, and a second type of ligand reversibly bound to the quantum dot; contacting the composition comprising the quantum dot (QD)-ligand conjugate with a tissue sample or a bodily fluid containing a pathogen to bond the QD-ligand conjugate to the pathogen and form a QD-pathogen complex, and subjecting the QD-pathogen complex to irradiation with a light source. In some instances, the first type of ligand is an antibody specific for a target pathogen and the second type of ligand quenches fluorescence of the QD while bound thereto. In some instances, the QD of the QD-ligand conjugate is configured to absorb light from a UV- or blue-emitting light source and emit light at a desired wavelength. In some instances, the desired wavelength of light is in the red portion of the electromagnetic spectrum. In some instances, the desired wavelength of light is in the green portion of the electromagnetic spectrum. In some instances, the desired wavelength of light is in the yellow portion of the electromagnetic spectrum. In some instances, the desired wavelength of light is in the orange portion of the electromagnetic spectrum. In some instances, the desired wavelength of light is in the violet portion of the electromagnetic spectrum. In some instances, the desired wavelength of light is in the infra-red portion of the electromagnetic spectrum. In some instances, the tissue sample is a population of white blood cells. In some instances, the tissue sample is a population of cells isolated from a bodily fluid. In some instances, the pathogen is selected from the group consisting of prions, viruses and microorganisms. In some instances, the pathogen is a coronavirus. In some instances, the pathogen is a coronavirus, wherein the coronavirus is SARS-CoV-2 or a strain or variant thereof. In some instances, the pathogen is a microorganism selected from the group consisting of bacteria, fungi, algae and parasites. In some instances, first type of ligand covalently bound to the quantum dot is anti-SARS-CoV-2 spike S1 protein. In some instances, first type of ligand covalently bound to the quantum dot is anti-SARS-CoV-2 spike S2 protein. In some instances, first type of ligand covalently bound to the quantum dot anti-SARS-CoV-2 nucleocapsid protein. In some instances, the composition is a gel formulation comprising the QD-ligand conjugate and a thickening agent. In some instances, the gel formulation further comprises at least one of a buffer and a gelling agent. In some instances, the QD-ligand conjugate is lyophilised prior to mixing with the tissue sample or bodily fluid. In some instances, the composition is a solution comprising the quantum dot (QD)-ligand conjugate and a solvent. In some instances, the solution further comprises at least one of a surfactant, a buffer and a stabilizing agent. In some instances, contacting the composition comprising the quantum dot (QD)-ligand conjugate with the tissue sample or the bodily fluid containing the pathogen comprises applying the composition on a surface where the tissue sample or bodily fluid is located. In some instances, methods according to the above can be performed using a lateral flow device.

### BRIEF DESCRIPTION OF THE DRAWINGS

FIG. 1 is a schematic illustration of a process for detecting a surface-mounted target (SARS-CoV-2 viral particles) using two colours of quantum dots by binding two different types of QD-ligand conjugates to the target, where the detected target is the resulting QD-target complex.

FIG. 2 is a fluorescence microscopy image showing biotinylated spheres labelled with red- and green-emitting QD-streptavidin conjugates overlapping to emit yellow light.

FIG. 3 is a schematic illustration of a process for detecting a surface-mounted target (SARS-CoV-2 viral particles) using antibody-quenched QDs by binding QD-ligand conjugates to the target, where the detected target is the resulting QD-target complex.

### DETAILED DESCRIPTION OF THE INVENTION

Herein, methods for the detection of surface-mounted biological systems and microorganisms using quantum dots are described.

The following description of the embodiments is merely exemplary in nature and is in no way intended to limit the subject matter of the disclosure, their application, or uses.

As used throughout, ranges are used as shorthand for describing each and every value that is within the range. Any value within the range can be selected as the terminus of the range. Unless otherwise specified, all percentages and amounts expressed herein and elsewhere in the specification should be understood to refer to percentages by weight.

For the purposes of this specification and appended claims, unless otherwise indicated, all numbers expressing quantities, percentages or proportions, and other numerical values used in the specification and claims, are to be understood as being modified in all instances by the term “about.” The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively ±5 percent, and alternatively ±1 percent of the given numeric value provided such a deviation does not alter the end function or result of the value. Accordingly, unless indicated to the contrary, the numerical parameters set forth in this specification and attached claims are approximations that can vary depending upon the desired properties sought to be obtained by the invention.

It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

As used herein, the term “biological system” or “pathogen” are used interchangeably and may include, but is not limited to, cells (for example, subtypes of white blood cells and isolated cells from bodily fluids), cellular tissues, prions, viruses, and microorganisms such as, but not limited to, bacteria, fungi, algae, and parasites.

As used herein, the term “nanoparticle” is used to describe a particle with dimensions on the order of approximately 1 to 100 nm. The term “quantum dot” (QD) is used to describe a semiconductor nanoparticle displaying quantum confinement effects. The dimensions of QDs are typically, but not exclusively, between about 1 to about 10 nm. According to various aspects of the disclosure, the dimension of QDs used herein may have dimensions of between about 1 to about 20 nm, alternatively between about 1 and about 15 nm. The terms “nanoparticle” and “quantum dot” are not intended to imply any restrictions on the shape of the particle.

QDs are fluorescent nanoparticles of semiconductor materials, with unique spectral properties including the ability to efficiently absorb light of a first wavelength and re-emit light of a second, longer wavelength, enabling use as surface-labelling agents.

Methods of synthesising core and core/shell nanoparticles are disclosed, for example, in co-owned U.S. Pat. Nos. 7,867,556, 7,867,557, 7,803,423, 7,588,828, and 6,379,635. The contents of each of the forgoing patents are hereby incorporated by reference, in their entirety. U.S. Pat. Nos. 9,115,097, 8,062,703, 7,985,446, 7,803,423, and 7,588,828, and U.S. Publication Nos. 2010/0283005, 2014/0264196, 2014/0277297 and 2014/0370690, the entire contents of each of which are hereby incorporated by reference, describe methods of producing large volumes of high quality monodisperse QDs.

A QD's compatibility with a medium as well as the QD's susceptibility to agglomeration, photo-oxidation and/or quenching, is mediated largely by the surface composition of the QD. The coordination about the final inorganic surface atoms in any core, core/shell or core/multi-shell nanoparticle may be incomplete, with highly reactive “dangling bonds” on the surface, which can lead to particle agglomeration. This problem is overcome by passivating (capping) the “bare” surface atoms with protecting organic groups, referred to herein as capping ligands or a capping agent. The capping or passivating of particles prevents particle agglomeration from occurring but also protects the particle from its surrounding chemical environment and provides electronic stabilisation (passivation) to the particles, in the case of core material. The capping ligand is usually a Lewis base bound to surface metal atoms of the outer most inorganic layer of the particle. The nature of the capping ligand largely determines the compatibility of the nanoparticle with a particular medium.

In many QD materials, the capping ligands are hydrophobic (for example, alkyl amines, alkyl thiols, fatty acids, alkyl phosphines, alkyl phosphine oxides, and the like). Thus, the QDs are typically dispersed in hydrophobic solvents, such as toluene, following synthesis and isolation of the hydrophobic ligand-capped quantum dots. Such hydrophobic capped QDs are typically not dispersible in more polar media. If surface modification of the QD is desired, the most widely used procedure is known as ligand exchange. Lipophilic ligand molecules that coordinate to the surface of the QD during core synthesis and/or shelling procedures may subsequently be exchanged with a polar/charged ligand compound. An alternative surface modification strategy intercalates polar/charged molecules or polymer molecules with the ligand molecules that are already coordinated to the surface of the QD. However, while certain ligand exchange and intercalation procedures render the QD more compatible with aqueous media, they may result in materials of lower photoluminescence quantum yield (QY) and/or substantially larger size than the corresponding unmodified nanoparticle. For certain applications, the QD is preferably substantially free of toxic elements such as cadmium, lead and arsenic (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of toxic elements such as cadmium, lead and arsenic) or is free of toxic elements such as cadmium, lead and arsenic. Examples of cadmium-, lead- and arsenic-free nanoparticles include nanoparticles comprising semiconductor materials, e.g., ZnS, ZnSe, ZnTe, InP, InN, InSb, AlN, AlP, AIS, AlSb, GaN, GaP, GaSb, CuInS₂, AgInS₂, AgInS₂/ZnS, S1, Ge, and alloys, graded alloys (such as, for example, InPZnS, InPZnSe and InPZnSe₍₁₋ₓ₎Sₓ), and doped derivatives thereof, particularly, nanoparticles comprising cores of one of these materials and one or more shells of another of these materials. In some circumstances, however, the use of QDs that contain toxic elements like Cd, As, Hg, or Pb is warranted for research purposes.

It is noted that QDs that include a single semiconductor material, e.g., ZnS, ZnSe, InP, GaN, etc. may have a relatively low quantum yield (QY) because of non-radiative electron-hole recombination that occurs at defects and dangling bonds at the surface of the QDs. In order to at least partially address these issues, the QD cores may be at least partially coated with one or more layers (also referred to herein as “shells”) of a material different than that of the core, for example a different semiconductor material than that of the “core.” The material included in the one or more shells may incorporate ions from any one or more of groups 2 to 16 of the periodic table. When a QD has two or more shells, each shell may be formed of a different material. In an exemplary core/shell material, the core is formed from one of the materials specified above and the shell includes a semiconductor material of larger band-gap energy and similar lattice dimensions as the core material. Exemplary shell materials include, but are not limited to, ZnS, ZnO, MgS, MgSe, MgTe and GaN. One example of a multi-shell QD is a core-shell-shell InP/ZnS/ZnO QD. The confinement of charge carriers within the core and away from surface states provides QDs of greater stability and higher QY.

However, while it is desirable to have QDs that lack heavy metals and other toxic elements, it has proved particularly difficult to modify the surface of cadmium-free QDs. Cadmium-free QDs readily degrade when methods such as the aforementioned ligand exchange methods are used to modify the surface of such cadmium-free QDs. For example, attempts to modify the surface of cadmium-free QDs have been observed to cause a significant decrease in the QY of such nanoparticles. For in vivo purposes, surface-modified cadmium-free QDs with high QY are required. For purposes of the invention, when referring to water-dispersible cadmium-free QDs: QY of <20% are considered very low; QY of <30% are considered low; QY of 30-40% are considered medium; QY>40% are considered high and QY>50% are considered very high.

The high QY cadmium-free water-dispersible QDs disclosed herein have a QY greater than about 20%. For certain in vivo embodiments, heavy metal-free semiconductor indium-based QDs or QDs containing indium and/or phosphorus are preferred. Non-limiting examples include: InP, and alloys of InPZnS, InPZnSe and InPZnSe₍₁₋ₓ₎Sₓ.

QDs used in accordance with varying aspects of the disclosure can have a size ranging from 1-15 nm before surface functionalisation. In some instances, the QDs can be core QDs. In some instances, the QDs can be alloyed core QDs. In some instances, the QDs can be a gradient alloyed core QDs. In some instances, the QDs can be core/shell QDs. In some instances, the QDs can be core/multi-shell QDs. QDs used in accordance with various aspects of the disclosure can be made of, or include, a core material comprising:

IIA-VIA (2-16) material, consisting of a first element from group 2 of the periodic table and a second element from group 16 of the periodic table and also including ternary and quaternary materials and doped materials. Nanoparticle material includes but is not restricted to: MgS, MgSe, MgTe, CaS, CaSe, CaTe, SrS, SrSe, SrTe, BaS, BaSe, BaTe;

IIB-VIA (12-16) material consisting of a first element from group 12 of the periodic table and a second element from group 16 of the periodic table and also including ternary and quaternary materials and doped materials. Nanoparticle material includes but is not restricted to: ZnS, ZnSe, ZnTe, CdS, CdSe, CdTe, HgS, HgSe, HgTe;

II-V material, consisting of a first element from group 12 of the periodic table and a second element from group 15 of the periodic table and also including ternary and quaternary materials and doped materials. Nanoparticle material includes but is not restricted to: Zn₃P₂, Zn₃N₂, Zn₃As₂, Cd₃P₂, Cd₃N₂, Cd₃As₂;

III-V material, consisting of a first element from group 13 of the periodic table and a second element from group 15 of the periodic table and also including ternary and quaternary materials and doped materials. Nanoparticle material includes but is not restricted to: BP, AlAs, AlN, AlP, AlSb, GaAs, GaN, GaP, GaSb, InAs, InN, InP, InSb, BN;

III-IV material, consisting of a first element from group 13 of the periodic table and a second element from group 14 of the periodic table and also including ternary and quaternary materials and doped materials. Nanoparticle material includes but is not restricted to: B₄C, Al₄C₃, Ga₄C;

III-VI material, consisting of a first element from group 13 of the periodic table and a second element from group 16 of the periodic table and also including ternary and quaternary materials. Nanoparticle material includes but is not restricted to: Al₂S₃, Al₂Se₃, Al₂Te₃, Ga₂S₃, Ga₂Se₃, GeTe, In₂S₃, In₂Se₃, Ga₂Te₃, In₂Te₃, InTe;

IV-VI material, consisting of a first element from group 14 of the periodic table and a second element from group 16 of the periodic table, and also including ternary and quaternary materials and doped materials. Nanoparticle material includes but is not restricted to: PbS, PbSe, PbTe, SnS, SnSe, SnTe;

V-VI material, consisting of a first element from group 15 of the periodic table and a second element from group 16 of the periodic table, and also including ternary and quaternary materials and doped materials. Nanoparticle material includes but is not restricted to: Bi₂Te₃, Bi₂Se₃, Sb₂Se₃, Sb₂Te₃;

Nanoparticle material, consisting of a first element from any group in the transition metal of the periodic table, and a second element from group 16 of the periodic table and also including ternary and quaternary materials and doped materials. Nanoparticle material includes but is not restricted to: NiS, CrS, CuInS₂, AgInS₂; and

Alloyed materials having III-V and IIB-VIA materials. Nanoparticle material includes but is not restricted to: InPZnS, InPZnSe and InPZnSe₍₁₋ₓ₎Sₓ.

By the term “doped QD,” for the purposes of specifications and claims, refers to QDs of the above and a dopant comprised of one or more main group or rare earth elements, this most often is a transition metal or rare earth element, such as but not limited to ZnS or InP QDs doped with Mn²⁺, Ca²⁺, Mg²⁺, and Al³⁺.

The term “ternary material,” for the purposes of specifications and claims, refers to QDs of the above but a three-component material. The three components are usually compositions of elements from the as mentioned groups, an example being (InₓGa₁₋ₓP)ₘLₙ QDs (where L is a capping agent).

The term “quaternary material,” for the purposes of specifications and claims, refers to QDs of the above but a four-component material. The four components are usually compositions of elements from the as mentioned groups, an example being (InPZnS)ₘLₙ QDs (where L is a capping agent).

The material used on any shell or subsequent numbers of shells grown onto the core particle in most cases will be of a similar lattice type material to the core material i.e. have close lattice match to the core material so that it can be epitaxially grown on to the core, but is not necessarily restricted to materials of this compatibility. The material used on any shell or subsequent numbers of shells grown on to the core present in most cases will have a wider bandgap than the core material but is not necessarily restricted to materials of this compatibility. The materials of any shell or subsequent numbers of shells grown on to the core can include material comprising:

IIA-VIA (2-16) material, consisting of a first element from group 2 of the periodic table and a second element from group 16 of the periodic table and also including ternary and quaternary materials and doped materials. Nanoparticle material includes but is not restricted to: MgS, MgSe, MgTe, CaS, CaSe, CaTe, SrS, SrSe, SrTe;

IIB-VIA (12-16) material, consisting of a first element from group 12 of the periodic table and a second element from group 16 of the periodic table and also including ternary and quaternary materials and doped materials. Nanoparticle material includes but is not restricted to: ZnS, ZnSe, ZnTe, CdS, CdSe, CdTe, HgS, HgSe, HgTe;

II-V material, consisting of a first element from group 12 of the periodic table and a second element from group 15 of the periodic table and also including ternary and quaternary materials and doped materials. Nanoparticle material includes but is not restricted to: Zn₃P₂, Zn₃N₂, Zn₃As₂, Cd₃P₂, Cd₃N₂, Cd₃As₂;

III-V material, consisting of a first element from group 13 of the periodic table and a second element from group 15 of the periodic table and also including ternary and quaternary materials and doped materials. Nanoparticle material includes but is not restricted to: BP, AlAs, AlN, AlP, AlSb, GaAs, GaN, GaP, GaSb, InAs, InN, InP, InSb, BN;

III-IV material, consisting of a first element from group 13 of the periodic table and a second element from group 14 of the periodic table and also including ternary and quaternary materials and doped materials. Nanoparticle material includes but is not restricted to: B₄C, Al₄C₃, Ga₄C;

III-VI material, consisting of a first element from group 13 of the periodic table and a second element from group 16 of the periodic table and also including ternary and quaternary materials. Nanoparticle material includes but is not restricted to: Al₂S₃, Al₂Se₃, Al₂Te₃, Ga₂S₃, Ga₂Se₃, In₂S₃, In₂Se₃, Ga₂Te₃, In₂Te₃;

IV-VI material, consisting of a first element from group 14 of the periodic table and a second element from group 16 of the periodic table and also including ternary and quaternary materials and doped materials. Nanoparticle material includes but is not restricted to: PbS, PbSe, PbTe, SnS, SnSe, SnTe;

V-VI material, consisting of a first element from group 15 of the periodic table and a second element from group 16 of the periodic table, and also including ternary and quaternary materials and doped materials. Nanoparticle material includes but is not restricted to: Bi₂Te₃, Bi₂Se₃, Sb₂Se₃, Sb₂Te₃; and

Nanoparticle material, consisting of a first element from any group in the transition metal of the periodic table, and a second element from group 16 of the periodic table and also including ternary and quaternary materials and doped materials. Nanoparticle material includes but is not restricted to: NiS, CrS, CuInS₂, AgInS₂.

The QDs may be conjugated with targeting ligands. As used herein, “targeting ligand” means a ligand having an affinity for the target pathogen. As used herein, “QD-ligand conjugate” means a QD that is surface-functionalised with a targeting ligand. Suitable targeting ligands can include, but are not restricted to: antibodies (including monoclonal or polyclonal antibodies); aptamers; and synthetic ligands with a specific affinity for the target antigen. In one example, biosynthetic ligands may be generated via biosynthesis using yeast (2-hybrid or n-hybrid systems) or phage display libraries. In another example, peptide ligands may be chemically synthesised.

Non-limiting examples of antibodies targeting specific diseases/organisms are summarised in Table 1.

TABLE 1

| Disease/Organism | Antibody |
|---|---|
| Blood count diseases | Anti-CD19 to detect B cells |
| Blood count diseases | Anti-CD3 to detect T cells |
| Prion diseases | anti-Human PRNP; PrP; Prion |
| | antibody |
| Pseudomonas aeruginosa | Pseudomonas aeruginosa monoclonal |
| | Antibody (B11) |
| Chlamydia | Anti-chlamydia trachomatis antibody |
| Brucellosis | Anti-brucella chimeric monoclonal |
| | antibody |
| Human papillomavirus | Anti-HPV18 L1 antibody |
| Protozoa | Anti-giardia lamblia antibody |
| | (ab28344) |
| Parasite leishmania donovani | Antibodies against leishmania |
| | donovani IgA positive control |
| Parasite schistosoma mansoni | antibodies against schistosoma |
| | mansoni IgG positive control |
| Plant pathogen xylella fastidiosa | Recombinant anti-X. fastidiosa |
| | antibody |
| Pathogenic fungi | Anti-fungus aspergillus antibody |
| Pathogenic algae protothecosis | Anti-prototheca zopfii antibodies |

In recent months, there has been widespread investigation into antibodies against COVID-19, the disease caused by the coronavirus SARS-CoV-2. Examples of antibodies against SARS-CoV-2 include anti-SARS-CoV-2 spike S1 protein; anti-SARS-CoV-2 spike S2 protein; anti-SARS-CoV-2 nucleocapsid; and other antibodies reported by Shi et al. [Nature, 2020, doi:10.1038/s41586-020-2381-y], and Ju et al. [Nature, 2020, doi: 10.1038/s4586-020-2380-z], which are hereby incorporated herein by reference in their entirety. One of ordinary skill in the art will appreciate that the disclosures provided herein may be applied to other coronavirus variants or strains.

The targeting ligands may be conjugated to the QDs via a covalent chemical bond, for example, by 1-ethyl-3-(3-dimethylaminopropyl)carbodiimide hydrochloride (EDC) chemistry. Other methods to conjugate targeting ligands to the QDs include, but are not restricted to, methods based on: biotin and streptavidin; proteins A and G; and lectins.

Once the QDs are functionalised with targeting ligands, they may be formulated into a preparation that can specifically bind to a corresponding target to form a QD-target (or QD-pathogen) complex that, when irradiated with a light source, may be visible to the naked eye or with a simple fluorescence camera. The specificity of this approach may be enhanced by using QDs emitting at two or more different wavelengths (colours) that can bind to the same target pathogen. When using two or more colours of QDs, the emitted light from each type (colour) of QDs will overlap to produce a unique combination of emitted light (that is, a unique emission fingerprint) that is specific and only attainable in the presence of a specific target pathogen. This dual colour approach eliminates the need to rinse excessive unbound QDs, a step that can result in the loss of substrate-bound targets. When using two or more types (colours) of QDs, the difference in the photoluminescence maximum (PLₘₐₓ) between the two types of QDs is at least 10 nm, preferably at least 25 nm, more preferably at least 50 nm, and more preferable at least 100 nm. For example, green-emitting QDs having a PLₘₐₓ at around 530 nm, a full-width at half-maximum (FWHM) of less than 60 nm and a QY greater than 30% may be used in combination with red-emitting QDs having a PLₘₐₓ at around 630 nm, an FWHM of less than 60 nm and a QY greater than 30%.

Alternatively, a single type (colour) of QDs may be conjugated with a first type of targeting ligand bound covalently to the QD surface and a second type of targeting ligand physically attached to (adsorbed on) the QD surface, the second type of targeting ligand being covalently linked with a QD quencher. Examples of suitable QD quenchers include, but are not restricted to: sexithiophenes; and 4-((4-(dimethylamino)phenyl)azo)benzoic acid (DABCYL). In the presence of a corresponding target, the first type of targeting ligand enables the QDs to latch on to the target, while the second type of targeting ligand becomes detached from the QD and binds to a different target, enabling the QDs to fluoresce upon excitation by a light source. The ability to switch the emission from the QDs on and off eliminates the need to rinse any unbound quenched QDs, a step that can cause loss of the substrate-bound targets.

Prior to application on a surface, the QD-ligand conjugates may be formulated into a preparation, to be applied to the surface. Suitable preparations include, but are not restricted to solutions or gels. Solutions may be delivered, for example, as a spray or aerosol or as prepared.

A solution may comprise a QD-ligand conjugate dissolved in a suitable solvent, such as, but not restricted to: water, binary solvents such as water-glycerol and water propylene glycol; aqueous buffers such as phosphate buffered saline (PBS), citrate or acetate buffers, buffers mixed with wetting agents such as glycerol or propylene glycol, or surfactants such as polysorbate 20; and buffers mixed with penetrating agents such as dimethyl sulfoxide (DMSO), 3-(3-cholamidopropyl)dimethylammonio-1-propanesulfonate (CHAPS) or a hydrate thereof, and digitonin. The solution may further comprise one or more additional components, including, but not restricted to: surfactant(s) such as polysorbate 20, Brij 60, and sodium dodecyl sulphate (SDS); buffer(s) such as phosphate, citrates, tris, 4-(2-hydroxyethyl)-1-piperazineethane sulphonic acid (HEPES), barbiturates, and acetate; stabilising agent(s) such as glycerol, betaine, propylene glycol, and amino acids; and the like. The concentration of QD-ligand conjugates within the final solution may be between about 10 μg/mL and about 100 μg/mL. A solution may be sprayed, coated or otherwise applied on a surface to detect a specific pathogen thereon.

A gel formulation may comprise a QD-ligand conjugate and a hydrophilic thickening agent. The gel formulation may further comprise one or more additional components, including, but not restricted to: buffer(s); and gelling agents such as pectin, gelatines, starch, dextran, cellulose derivatives, polyacrylic acids, polyvinylpyrrolidone (PVP), and polyvinyl acetate (PVA). In some instances, the hydrophilic thickening agent may provide thixotropic (shear thinning) properties, which are desirable to enable packaging as a rigid gel that softens upon rubbing into a rigid surface. Examples of suitable hydrophilic thickening agents include, but are not restricted to: polyacrylic acid; microcrystalline cellulose; hydroxypropylmethyl cellulose; and polyethylene glycol (PEG). The concentration of QD-ligand conjugates in the gel may be between about 10 μg/mL and about 1,000 μg/m L. The gel may be applied to a surface and rubbed in to detect a specific pathogen thereon. The static viscosity of the gel at room temperature may be between about 300 cps and about 3,000 cps, more preferably between about 1,000 cps and about 2,000 cps.

Alternatively, the QD-ligand conjugates can be lyophilised in the presence of a suitable carrier excipient, which is loaded into a depot of a lateral flow device. The lateral flow device comprises a paper strip printed with predesigned line(s) or spot(s) of capturing antibodies against the target epitome(s). The strip is dipped into a test sample, such as washings from an examined surface, or a bodily fluid such as urine, sputum, or saliva, and subsequently the QD-ligand conjugates are released from the depot onto the strip using a buffer and, via capillary action, the QD-ligand conjugates reach the capillary line(s)/spot(s) on the strip and bind to the target if it is captured. Suitable carrier excipients include, but are not restricted to mannitol, sorbitol, sucrose, inorganic salts, amino acids, and surfactants. Suitable buffers include, but are not restricted to citrate buffer (pH 5-6), acetate buffer (pH 5-7), and HEPES buffer (pH 6-7).

For the detection of viruses, testing methods based on a single antibody can be prone to false positive results due to crosstalk with other types of virus. The accuracy of the lateral flow detection system may be improved by using QDs emitting at two or more different wavelengths (colours) that can bind to the same target pathogen. For example, a first population of QDs emitting at a first wavelength (colour) may be conjugated with a first type of conjugating ligand targeting a first structural protein on a virus and a second population of QDs emitting at a second wavelength (colour) may be conjugated with a second type of conjugating ligand targeting a second structural protein on the same virus, allowing the first and second population of QD-ligand conjugates to bind to the virus particles in a sample. The presence of the virus may subsequently be detected from the combined fluorescence of the QDs emitting at the first wavelength (colour) and the QDs emitting at the second wavelength (colour) on the testing strip when irradiated with a light source. The unique emission will occur only in the simultaneous presence of the two targeted viral proteins. By simultaneously detecting two viral proteins, the detection system may enable the detection of the whole virus, rather than fragments, avoiding false positive results.

In some embodiments, a plurality of QD-ligand conjugates, each targeting a different pathogen, are combined in a single preparation or detection system. The unique emission fingerprint generated by each QD-ligand conjugate can be used to distinguish between two or more different pathogens, for example, two or more different viruses (e.g. a coronavirus such as SARS-CoV-2 and influenza, respiratory syncytial virus (RSV), and/or adenovirus), or two or more different strains or variants of the same pathogen. In some embodiments, the unique emission fingerprint generated by each QD-ligand conjugate can be used to distinguish between two different pathogens that require different methods of treatment, for example, a virus and a bacterial infection. Thus, the result of the test can be used to influence the therapy administered. For example, accurately identifying whether symptoms are caused by a virus or bacterium may avoid the inappropriate use of antibiotics.

In some embodiments, the QDs are impregnated into beads of a high reflectance material in order to enhance their emission signal. Suitable high reflectance materials include, but are not restricted to: barium sulphate; and polytetrafluoroethylene (PTFE). For example, QDs can be incorporated into PTFE beads using a solvent swelling and soaking method. The QD-impregnated beads can subsequently be surface-modified to provide water solubility, followed by functionalisation with targeting ligands to form QD bead-ligand conjugates.

In some embodiments, the compositions containing QD-ligand conjugates according to the disclosure may further comprise a disinfecting agent, to simultaneously detect and destroy a pathogen. A suitable disinfecting agent is capable of destroying the pathogen, without damaging the QDs and/or the targeting ligand conjugated thereto.

A suitable light source for the excitation of QDs may include a source of light having a wavelength shorter than the emission wavelength of the QDs. Examples include, but are not restricted to, a UV- or blue-emitting light source such as a UV- or blue-emitting light-emitting diode (LED).

QDs offer a number of advantages over fluorescent dyes for the detection of surface-mounted pathogens. The high absorption coefficient of QDs facilitates the use of an intense excitation source and achieves stronger emission than that from small organic fluorophores. This stronger emission can be detected by the naked eye or a simple camera detector. Furthermore, the use of an intense excitation source on surfaces can lead to reflectance, such that if the excitation wavelength is close to the emission wavelength, as is the case for conventional fluorescent dye molecules, the signal from the emitted light cannot be separated from the reflectance signal. In the case of QDs, the excitation wavelength is broad and can be deconvoluted from reflectance interference.

### Example 1: Preparation of Non-Toxic QDs

A molecular seeding process was used to generate non-toxic QDs. Briefly, the preparation of non-functionalised indium-based QDs with emission in the range of 500-720 nm was carried out as follows: dibutyl ester (approximately 100 mL) and myristic acid (MA) (10.06 g) were placed in a three-neck flask and degassed at about 70° C. under vacuum for 1 h. After this period, nitrogen was introduced and the temperature was increased to about 90° C. Approximately 4.7 g of a ZnS molecular cluster ([Et₃NH]₄[Zn₁₀S₄(SPh)₁₆]) was added, and the resulting mixture was stirred for approximately 45 min. The temperature was then increased to about 100° C., followed by the dropwise additions of indium myristate In(MA)₃ (1M; 15 mL) followed by tris-trimethylsilyl phosphine (TMS)₃P (1M; 15 mL). The reaction mixture was stirred while the temperature was increased to about 140° C. At 140° C., further dropwise additions of In(MA)₃ dissolved in di-n-butylsebacate ester (1M; 35 mL) (left to stir for 5 min) and (TMS)₃P dissolved in di-n-butylsebacate ester (1M; 35 mL) were made. The temperature was then slowly increased to 180° C., and further dropwise additions of In(MA)₃ (1M; 55 mL) followed by (TMS)₃P (1M; 40 mL) were made. By addition of the precursor in this manner, indium-based particles with a PLₘₐₓ gradually increasing from 500 nm to 720 nm were formed. The reaction was stopped when the desired emission maximum was obtained and left to stir at the reaction temperature for half an hour. After this period, the mixture was left to anneal for up to approximately 4 days (at a temperature ˜20-40° C. below that of the reaction). A UV lamp was also used at this stage to aid in annealing.

The resulting particles were isolated by the addition of dried degassed methanol (approximately 200 mL) via cannula techniques. The precipitate was allowed to settle and then methanol was removed via cannula with the aid of a filter stick. Dried degassed chloroform (approximately 10 mL) was added to wash the solid. The solid was left to dry under vacuum for 1 day. This procedure resulted in the formation of indium-based nanoparticles on ZnS molecular clusters. In further treatments, the QYs of the resulting indium-based nanoparticles were further increased by washing in dilute hydrofluoric acid (HF). The QY of the indium-based core material ranged from approximately 25%-50%. This composition is considered an alloy structure comprising In, P, Zn and S.

Growth of a ZnS shell: A 20 mL portion of the HF-etched indium-based core particles was dried in a three-neck flask. 1.3 g of myristic acid and 20 mL di-n-butyl sebacate ester were added and degassed for 30 min. The solution was heated to 200° C., and 2 mL of 1 M (TMS)₂S was added dropwise (at a rate of 7.93 mL/h). After this addition was complete, the solution was left to stand for 2 min, and then 1.2 g of anhydrous zinc acetate was added. The solution was kept at 200° C. for 1 h and then cooled to room temperature. The resulting particles were isolated by adding 40 mL of anhydrous degassed methanol and centrifuging. The supernatant liquid was discarded, and 30 mL of anhydrous degassed hexane was added to the remaining solid. The solution was allowed to settle for 5 h and then centrifuged again. The supernatant liquid was collected and the remaining solid was discarded. The QYs of the final non-functionalised indium-based nanoparticle material ranged from approximately 60%-90% in organic solvents.

### Example 2: Synthesis of Water-Soluble Surface-Modified QDs

Provided herein is one embodiment of a method for generating and using hexamethoxymethylmelamine (HMMM)-modified fluorescent QDs as drug delivery vehicles. The unique melamine-based coating presents excellent biocompatibility, low toxicity and very low non-specific binding. These unique features allow a wide range of biomedical applications both in vitro and in vivo.

One example of preparation of a suitable water-soluble QDs is provided as follows: 200 mg of cadmium-free QDs with red emission at 608 nm having as a core material an alloy comprising indium and phosphorus with Zn-containing shells as described in Example 1 was dispersed in toluene (1 mL) with isopropyl myristate (100 μL). The isopropyl myristate is included as a ligand interactive agent. The mixture was heated at 50° C. for about 1-2 minutes then slowly shaken for 15 hours at room temperature. A toluene solution (4 mL) of HMMM (CYMEL 303, available from Cytec Industries, Inc., West Paterson, N.J.) (400 mg), monomethoxy polyethylene oxide (CH3O-PEG2000-OH) (400 mg), and salicylic acid (50 mg) was added to the nanoparticle dispersion. The salicylic acid that is included in the functionalisation reaction plays three roles: as a catalyst, a crosslinker, and a source for COOH. Due in part to the preference of HMMM for OH groups, many COOH groups provided by the salicylic acid remain available on the QD after crosslinking.

HMMM is a melamine-based linking/crosslinking agent having the following structure:

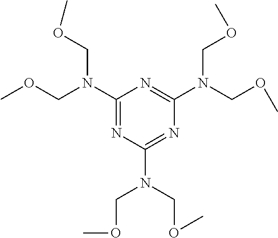

HMMM can react in an acid-catalysed reaction to crosslink various functional groups, such as amides, carboxyl groups, hydroxyl groups, and thiols.

The mixture was degassed and refluxed at 130° C. for the first hour followed by 140° C. for 3 h while stirring at 300 rpm with a magnetic stirrer. During the first hour, a stream of nitrogen was passed through the flask to ensure the removal of volatile by-products generated by the reaction of HMMM with nucleophiles. The mixture was allowed to cool to room temperature and stored under inert gas. The surface-modified QDs showed little or no loss in fluorescence QY and no change in the PLₘₐₓ or FWHM value, compared to unmodified QDs. An aliquot of the surface-modified nanoparticles was dried under vacuum and deionised water was added to the residue. The surface-modified nanoparticles dispersed well in the aqueous media and remained dispersed permanently. In contrast, unmodified nanoparticles could not be suspended in the aqueous medium. The fluorescence QY of the surface-modified nanoparticles according to the above procedure is 40-50%. In typical batches, a quantum yield of 47%±5% is obtained.

In another embodiment, cadmium-free QDs (200 mg) with red emission at 608 nm were dispersed in toluene (1 mL) with cholesterol (71.5 mg). The mixture was heated at 50° C. for about 1-2 minutes then slowly shaken for 15 h at room temperature. A toluene solution (4 mL) of HMMM (CYMEL 303) (400 mg), monomethoxy polyethylene oxide (CH₃O-PEG₂₀₀₀-OH) (400 mg), guaifenesin (100 mg), dichloromethane (DCM) (2 mL) and salicylic acid (50 mg) was added to the QD dispersion.

As used herein the compound “guaifenesin” has the following chemical structure:

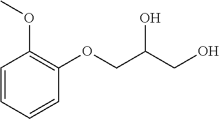

As used herein the compound “salicylic acid” has the following chemical structure:

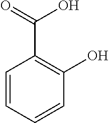

The mixture was degassed and refluxed at 140° C. for 4 hours while stirring at 300 rpm with a magnetic stirrer. As with the prior procedure, during the first hour a stream of nitrogen was passed through the flask to ensure the removal of volatile by-products generated by the reaction of HMMM with nucleophiles. The mixture was allowed to cool to room temperature and stored under inert gas. An aliquot of the surface-modified QDs was dried under vacuum and deionised water was added to the residue. The pH of the solution was adjusted to 6.5 using a 100 mM KOH solution and the excess non reacted material was removed by three cycles of ultrafiltration using Amicon filters (30 kD). The final aqueous solution was kept refrigerated until use.

It is noteworthy that traditional methods for modifying QDs to increase their water solubility (e.g., ligand exchange with mercapto-functionalised water-soluble ligands) are ineffective under mild conditions to render the QDs water soluble. Under harsher conditions, such as heat and sonication, the fraction that becomes water-soluble has very low QY (<20%). The instant method, in contrast, provides water-soluble QDs with high QY. As defined herein, a high QY is equal to or greater than 40%. In certain embodiments, a high QY is obtained of equal to or greater than 45%. The surface-modified QDs prepared as in this example also disperse well and remain permanently dispersed in other polar solvents, including ethanol, propanol, acetone, methylethylketone, butanol, tripropylmethylmethacrylate, or methylmethacrylate.

### Example 3: Preparation of QD-Ligand Conjugates

An activation buffer having a pH of 4.5 was prepared by mixing 2-(N-morpholino)ethanesulphonic acid (MES; 25 mM) in deionised water. In an Eppendorf tube, 2.5 mg of the water-soluble QDs prepared in Example 2 were mixed with activation buffer (about 100 μL). 1-ethyl-3-(3-dimethylaminopropyl)carbodiimide hydrochloride (EDC) solution (33 μL; 33 mg/mL) was added and the solution was mixed. N-hydroxysulfosuccinimide (sulfo-NHS) solution in deionised water (4 μL; 100 mg/mL) was added, followed by further mixing. The QD/MES/EDC/sulfo-NHS solution was added to an Amicon 30 kD centrifugation filter, then topped up to 2,000 μL with MES, followed by centrifugation at 2,500 rcf for 30 min. The retained QDs were re-dispersed in activation buffer (60 μL), then transferred to an Eppendorf tube containing 10 μL of an antibody (100 mg/mL in phosphate-buffered saline (PBS) stock solution) and 4-(2-hydroxyethyl)-1-piperazineethane sulphonic acid (HEPES; 40 μL) at pH 8.5. The solution was mixed well, then incubated overnight (16-18 h) at room temperature. The solution was quenched with a solution of 6-aminocaproic acid (6AC; 16 μL; 100 mM in deionised water). To purify the QD-ligand conjugates, the solution was transferred to a Nanosep 300K filter, pre-whetted with 100 μL PBS, then topped up to the 500 μL line with further PBS. Excess antibody was removed via three cycles of ultrafiltration using Nanosep 300K filters and 100 μL PBS buffer. For each cycle, centrifugation was carried out at 2,000 rcf for 30 min, followed by re-dispersion in ˜400 μL PBS. The final QD-ligands conjugates were re-dispersed in 100 μL PBS.

### Example 3.1: Preparation of QD-Anti-SARS-CoV-2 Spike S1 Conjugates

An activation buffer having a pH of 4.5 was prepared by mixing MES (25 mM) in deionised water. In an Eppendorf tube, 2.3 mg of water-soluble heavy metal-free QDs (PLₘₐₓ=635 nm; FWHM=54 nm; QY=42%) were mixed with activation buffer (˜100 μL). EDC solution (33 μL; 33 mg/mL) was added and the solution was mixed. Sulfo-NHS solution in deionised water (4 μL; 100 mg/mL) was added, followed by further mixing, then left to equilibrate and activate prior to cleaning. The QD/MES/EDC/sulfo-NHS solution was added to an Amicon 30 kD centrifugation filter, then topped up to 4,000 μL with MES, followed by centrifugation at 3,000 rcf for 30 min. The QDs were diluted 10× using MES, then transferred to an Eppendorf tube containing 0.1 mg of anti-SARS-CoV-2 spike S1 made up to 50 μL using HEPES buffer. The solution was mixed well, then incubated for 30 min at room temperature. The solution was then placed in a 37° C. water bath in a polystyrene float for 30 min, then placed in a fridge at 4° C. overnight. The solution was quenched with a solution of 6-am inocaproic acid (6AC; 8 μL; 100 mM in deionised water). To purify the QD-ligand conjugates, the solution was transferred to an Amicon 100 kD centrifugation filter, pre-whetted with 100 μL PBS, then topped up to the 500 μL line with further PBS. This was spun at 5,200 rpm for 30 min to concentrate the sample (with a sequential 2,500 rpm spin for 5 min with the filter reversed to collect the sample). Excess antibody was removed using a size exclusion gel column. The sample was removed and about 50 μL was added per gel column, then allowed to stand for 5 min. 4 mL PBS was added to the gel column. The majority of the buffer was allowed to run through. When the sample was about 1 cm from the bottom of the gel, fractions (3-4 drops) were collected using Eppendorf tubes until all QD-ligand conjugates had been collected.

### Example 3.2: Preparation of QD-Anti-SARS-CoV-2 Nucleocapsid Conjugates

An activation buffer having a pH of 4.5 was prepared by mixing MES (25 mM) in deionised water. In an Eppendorf tube, 2.3 mg of water-soluble heavy metal-free QDs (PLₘₐₓ=635 nm; FWHM=54 nm; QY=42%) were mixed with activation buffer (˜100 μL). EDC solution (33 μL; 33 mg/mL) was added and the solution was mixed. Sulfo-NHS solution in deionised water (4 μL; 100 mg/mL) was added, followed by further mixing. The QD/MES/EDC/sulfo-NHS solution was added to an Amicon 30 kD centrifugation filter, then topped up to 4,000 μL with MES, followed by centrifugation at 3,000 rcf for 30 min. The QDs were diluted 10× using MES, then transferred to an Eppendorf tube containing 0.1 mg of anti-SARS-CoV-2 spike S1 made up to 50 μL using HEPES buffer. The solution was mixed well, then incubated for 30 min at room temperature. The solution was then placed in a 37° C. water bath in a polystyrene float for 30 min, then placed in a fridge at 4° C. overnight. The solution was quenched with a solution of 6-aminocaproic acid (6AC; 8 μL; 100 mM in deionised water). To purify the QD-ligand conjugates, the solution was transferred to an Amicon 100 kD centrifugation filter, pre-whetted with 100 μL PBS, then topped up to the 500 μL line with further PBS. This was spun at 5,200 rpm for 30 min to concentrate the sample (with a sequential 2,500 rpm spin for 5 min with the filter reversed to collect the sample). Excess antibody was removed using a size exclusion gel column. The sample was removed and about 50 μL was added per gel column, then allowed to stand for 5 min. 4 mL PBS was added to the gel column. The majority of the buffer was allowed to run through. When the sample was about 1 cm from the bottom of the gel, fractions (3-4 drops) were collected using Eppendorf tubes until all QD-ligand conjugates had been collected.

### Example 4: Preparation of a QD-Ligand Conjugate Solution for Pathogen Detection

QD-ligand conjugates as prepared in Example 3 (at a concentration between 10-100 mg/mL) were combined with NaCl (20 mg/mL), sodium citrate (5 mg/mL), and polyethylene glycol sorbitan monolaurate (TWEEN® 20 surfactant available from Sigma-Aldrich, UK; 1 mg/mL). The pH was adjusted to 6 using KOH, then the solution was diluted 1,000-fold with deionised water before use.

### Example 5: Preparation of a QD-Ligand Conjugate Gel for Pathogen Detection

QD-ligand conjugates as prepared in Example 3 (at a concentration between 10-1,000 μg/mL) were combined with NaCl (20 mg/mL) and sodium citrate (5 mg/mL), adjusted to pH 6 using KOH, then mixed with a hydrophilic thickening agent having thixotropic properties to form a gel.

### Example 6: Preparation of a Lateral Flow Device

QD-ligand conjugates as prepared in Example 3 were lyophilised in the presence of an excipient carrier and loaded into the depot of a lateral flow device. The paper strip of the device was printed with predesigned strips or spots of capturing antibodies against the target epitome. The strip was dipped into a test sample, then the QD-ligand conjugates were released from the depot into the strip using citrate buffer at pH 6. Via capillary action, the QD-ligand conjugates reached the target lines or spots on the strip and bound to the target if the correct target was present.

### Example 7. Detection of Surface-Mounted Targets Using Two Colours of Quantum Dots

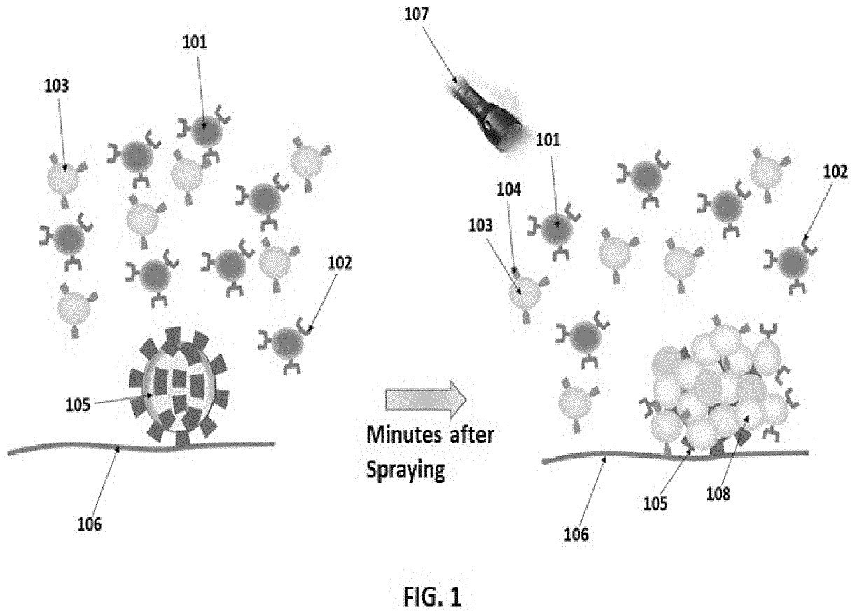

FIG. 1 illustrates a process for detecting a surface-mounted target using two colours of QDs. Red-emitting QDs (101; for example VIVODOTS® 630 nanoparticles available from Nanoco Technologies Limited, Manchester, UK, emitting with a PLₘₐₓ centred around 630 nm) conjugated to a first antibody (102; e.g. anti-SARS-CoV-2 spike S1 protein) and green-emitting QDs (103; for example VIVODOTS™ 530 nanoparticles available from Nanoco Technologies Limited, Manchester, UK, emitting with a PLₘₐₓ centred around 530 nm) conjugated to a second antibody (104; e.g. anti SARS-CoV-2 spike S2 protein) are applied to a surface (106) contaminated with spots of the pathogen for COVID-19, SARS-CoV-2 virus (105). The red- and green-emitting QDs bound to the anti-SARS-CoV-2 antibodies can accumulate on the SARS-CoV-2 viral particles. When illuminated with a short wavelength emission source (107) such as, for example, a blue light source (e.g. emitting around 400 nm), the red- and green-emitting QDs accumulating on the SARS-CoV-2 viral particles can, when irradiated with a UV-of blue-emitting light source, produce yellow light (108) generated from the overlap between the green and red emission from the QDs. Unbound QDs remain fluorescent (red or green) but with lower fluorescence intensity.

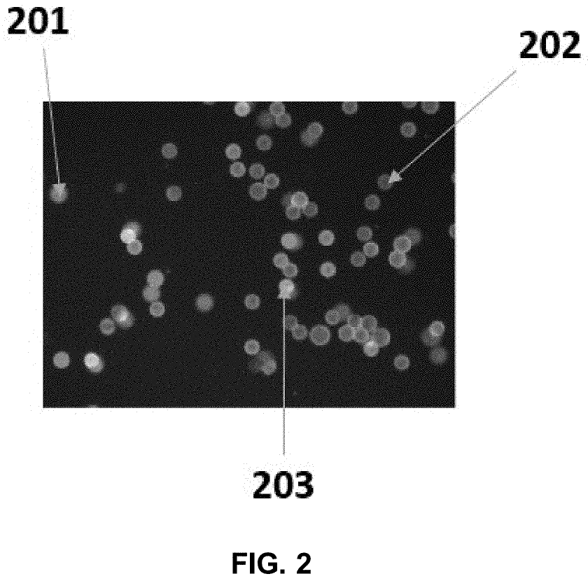

The process of overlapping emission from red- and green-emitting QDs to produce yellow light is demonstrated in the microscopy image in FIG. 2, wherein biotinylated spheres labelled with red-emitting QDs conjugated to streptavidin (201) and biotinylated spheres labelled with green-emitting QDs conjugated to streptavidin (202) overlapped to produce yellow-emitting spheres (203).

### Example 8. Detection of Surface-Mounted Targets Using Antibody-Quenched QDs

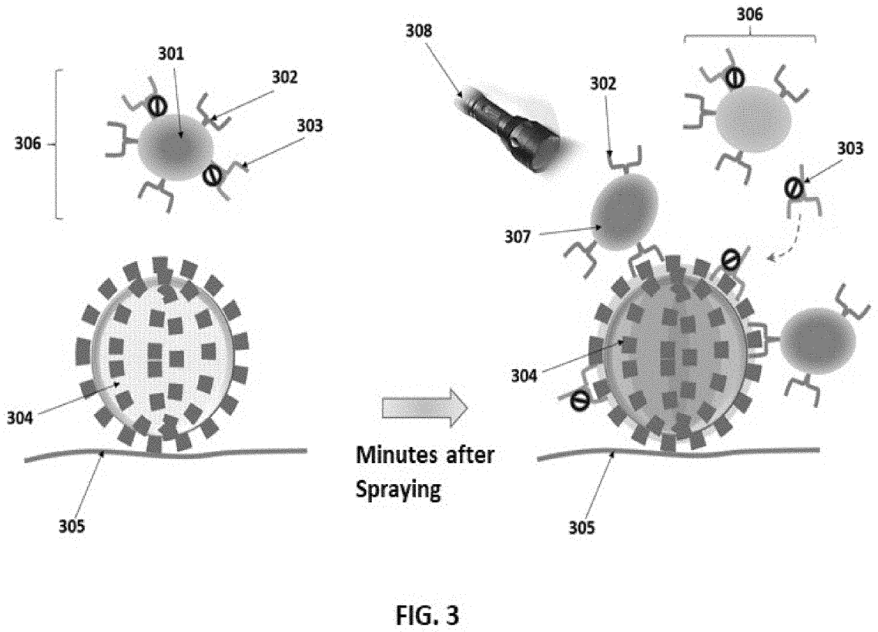

FIG. 3 illustrates a process for detecting a surface-mounted target using antibody-quenched QDs. To form quenched QDs (306), QDs (301; e.g. red-emitting VIVODOTS® 630 nanoparticles available from Nanoco Technologies Limited, Manchester, UK, emitting with a PLₘₐₓ centred around 630 nm) are conjugated with two types of antibodies (302 and 303). The first type of antibody (302; e.g. anti-SARS-CoV-2 spike S1 protein) is an antibody without a quencher and is covalently and irreversibly attached to the QD. The second type of antibody (303) is an antibody against a different type of protein (e.g. anti-SARS-CoV-2 spike S2 protein) and is covalently linked to a QD quencher. The quencher antibody (303) is physically and reversibly attached to the QD using hydrophobic interaction forces. When not in contact with a target pathogen such as SARS-CoV-2, the quenched QDs (301) are non-emissive. Upon reaching the target pathogen (304) bound to a surface (305), the first antibody (302) can enable the quenched QD (306) to latch onto the target pathogen (304) and the second antibody (303) will leave the QD (301) and bind to a different target, resulting in fluorescent QDs (307) upon illumination by a short wavelength excitation source (308; for example, a blue LED emitting around 400 nm), causing the target to fluoresce.

The methods for the detection of surface-mounted biological samples and pathogens as described herein may be applicable not just in healthcare setting, but also in the field of exposure, in the home environment, in veterinary settings, and in agricultural settings (e.g. the detection of infections to crops).

The methods described herein offer a number of advantages over conventional methods disclosed in the prior art. For example, a spray or gel as described herein may be used to detect biological samples or pathogens on a surface more quickly and easily, using the naked eye or a simple fluorescent camera, than a lateral flow system. The dual-colour embodiment described herein may be applied to a lateral flow system to offer advantages over conventional, single-colour lateral flow systems, which may be prone to false positive results.

The foregoing presents particular embodiments embodying the principles of the invention. Those skilled in the art will be able to devise alternatives and variations which, even if not explicitly disclosed herein, embody those principles and are thus within the scope of the invention. Although particular embodiments of the present invention have been shown and described, they are not intended to limit what this patent covers. One skilled in the art will understand that various changes and modifications may be made without departing from the scope of the present invention as literally and equivalently covered by the following claims.

What is claimed is:

1. A method for detecting a pathogen in a tissue sample or bodily fluid, the method comprising,
providing a composition comprising a population of quantum dot (QD)-ligand conjugates, the population of QD-ligand conjugates comprising:
a first population of QD-ligand conjugates configured to absorb light from a UV-of blue-emitting light source and emit light at a first wavelength; and
a second population of QD-ligand conjugates configured to absorb light from a UV- or blue-emitting light source and emit light at a first wavelength;
contacting the composition comprising the population of quantum dot (QD)-ligand conjugates with a tissue sample or a bodily fluid containing a pathogen to bond one or more of the QD-ligand conjugates to the pathogen and form QD-pathogen complexes, and
subjecting the QD-pathogen complexes to irradiation with a light source.

2. The method of claim 1, wherein the tissue sample is a population of white blood cells.

3. The method of claim 1, wherein the tissue sample is a population of cells isolated from a bodily fluid.

4. The method of claim 1, wherein the pathogen is selected from the group consisting of prions, viruses and microorganisms.

5. The method claim 4, wherein the virus is a coronavirus.

6. The method of claim 5, wherein the coronavirus is SARS-CoV-2 or a strain or variant thereof.

7. The method of claim 4, wherein the microorganism is any selected from the group consisting of bacteria, fungi, algae and parasites.

8. The method of claim 1, wherein a QD-ligand conjugate of the first population comprises a QD conjugated to anti-SARS-CoV-2 spike S1 protein and a QD-ligand conjugate of the second population comprises a QD conjugated to anti-SARS-CoV-2 nucleocapsid protein.

9. The method of claim 1, wherein the first wavelength of light is in the red portion of the electromagnetic spectrum and/or the second wavelength of light is in the green portion of the electromagnetic spectrum.

10. The method of claim 1, wherein
the QD-ligand conjugates of the first population are made of red-emitting conjugates conjugated to anti-SARS-CoV-2 spike S1 protein and the QD-ligand conjugates of the second population are made of green-emitting conjugates conjugated to anti-SARS-CoV-2 spike S2 protein; or
wherein the QD-ligand conjugates of the first population are made of green-emitting conjugates conjugated to anti-SARS-CoV-2 spike S1 protein and the QD-ligand conjugates of the second population are made of red-emitting conjugates conjugated to anti-SARS-CoV-2 spike S2 protein.

11. The method of claim 10, wherein yellow light is produced when QD-ligand conjugates of the first population and QD-ligand conjugates of the second population are bound to SARS-CoV-2 virus and subjected to irradiation with a UV-of blue-emitting light source.

12. The method of claim 1, wherein the composition is a gel formulation comprising the population of QD-ligand conjugates and a thickening agent.

13. The method of claim 12, wherein the gel formulation further comprises at least one of a buffer and a gelling agent.

14. The method of claim 1, wherein the population of QD-ligand conjugates is lyophilised prior to mixing with the tissue sample or bodily fluid.

15. The method of claim 1, wherein the composition is a solution comprising the population of quantum dot (QD)-ligand conjugates and a solvent.

16. The method of claim 1, wherein the solution further comprises at least one of a surfactant, a buffer and a stabilizing agent.

17. The method of claim 1, wherein contacting the composition comprising the population of quantum dot (QD)-ligand conjugates with the tissue sample or the bodily fluid containing the pathogen comprises applying the composition on a surface where the tissue sample or bodily fluid is located.

18. The method of claim 1, wherein the method is performed using a lateral flow device.

19. A method for detecting a pathogen in a tissue sample or bodily fluid, the method comprising,
providing a composition comprising a quantum dot (QD)-ligand conjugate, the QD-ligand conjugate comprising:
a first type of ligand covalently bound to the quantum dot; and
a second type of ligand reversibly bound to the quantum dot;
contacting the composition comprising the quantum dot (QD)-ligand conjugate with a tissue sample or a bodily fluid containing a pathogen to bond the QD-ligand conjugate to the pathogen and form a QD-pathogen complex, and
subjecting the QD-pathogen complex to irradiation with a light source.

20. The method of claim 19, wherein the first type of ligand is an antibody specific for a target pathogen and the second type of ligand quenches fluorescence of the quantum dot while bound thereto.
